(19) United States
(12) Patent Application Publication
(10) Pub. No.: US 2014/0005785 A1
(43) Pub. Date: Jan. 2, 2014

(54) LINEAR ELECTROMECHANICAL DEVICE-BASED ARTIFICIAL MUSCLES, BIO-VALVES AND RELATED APPLICATIONS

(71) Applicant: Orsen LLC, Albuquerque, NM (US)

(72) Inventors: Richard J. Massen, Albuquerque, NM (US); Luis M. Ortiz, Albuquerque, NM (US)

(73) Assignee: Orsen LLC, Albuquerque, NM (US)

(21) Appl. No.: 14/026,186

(22) Filed: Sep. 13, 2013

Related U.S. Application Data
(62) Division of application No. 11/219,997, filed on Sep. 6, 2005, now abandoned.
Application No. 11/219,997 is a continuation-in-part of application No. 11/007,457, filed on Dec. 9, 2004.
Application No. 11/007,457 is a continuation-in-part of application No. 10/923,357, filed on Aug. 20, 2004, now abandoned.

(51) Int. Cl. A61F 2/08

(52) U.S. Cl. CPC A61F 2/08
USPC 623/14.13

(57) ABSTRACT

A biological function assist apparatus composed of a linear electronmechanical device or system wrapped in protective coating and controlled by a controller, which also provides power to the electromechanically-based system. The electromechanically-based system can be formed as a mesh using linear motors or linear actuators or a larger electromechanically grid and wrapped around a failing heart. The electromechanical system can be formed in a circle forming an artificial valve (e.g., sphincter). The electromechanically-based system can also operate as a bone-muscle interface, thereby functioning in place of tendons.

### APPLICATION PRIORITY

The present application is related to and claims priority as a Divisional of U.S. application Ser. No. 11/219,997, filed Sep. 6, 2005, entitled “Linear electromechanical device-based artificial muscles, bio-valves and related applications,” which is a Continuation-in-Part of application Ser. No. 11/007,457, filed Dec. 9, 2004, entitled “Electromechanical Machine-based Artificial Muscles, Bio-Valves and related devices”, which is a Continuation-in-Part of U.S. patent application Ser. No. 10/923,357, entitled “Micro electromechanical machine-based ventricular assist apparatus,” filed on Aug. 20, 2004. All prior applications are hereby incorporated by reference herein in their entirety.

### FIELD OF THE INVENTION

Embodiments are generally related to electromechanical systems. The embodiments are also related to artificial muscles. More particularly, embodiments are related to linear electromechanical-based devices useful for biomedical application such as artificial muscles, bio-valves and related devices. Embodiments are also related to devices for assisting natural human organs and body parts assisted by linear electromechanical devices and systems.

### BACKGROUND

The natural human heart and accompanying circulatory system are critical components of the human body and systematically provide the needed nutrients and oxygen for the body. As such, the proper operation of a circulatory system, and particularly, the proper operation of the heart, is critical in the overall health and well being of a person. A physical ailment or condition which compromises the normal and healthy operation of the heart can therefore be particularly critical and may result in a condition which must be medically remedied.

Specifically, the natural heart, or rather the cardiac tissue of the heart, can fail for various reasons to a point where the heart can no longer provide sufficient circulation of blood for the body so that life can be maintained. To address the problem of a failing natural heart, conventional solutions have been offered to provide techniques for which circulation of blood might be maintained.

Some solutions involve replacing the heart. Other solutions maintain the operation of the existing heart. One such solution has been to replace the existing natural heart in a patient with an artificial heart or a ventricular assist device. In utilizing artificial hearts and/or assist devices, a particular problem stems from the fact that the materials used for the interior lining of the chambers of an artificial heart are in direct contact with the circulating blood. Such contact may enhance the undesirable clotting of the blood, may cause a build-up of calcium, or may otherwise inhibit the blood's normal function. As a result, thromboembolism and hemolysis may occur.

Additionally, the lining of an artificial heart or a ventricular assist device can crack, which inhibits performance, even when the crack is at a microscopic level. Moreover, these devices must be powered by a power source, which may be cumbersome and/or external to the body. Such drawbacks have limited use of artificial heart devices to applications having too brief of a time period to provide a real lasting benefit to the patient.

An alternative procedure also involves replacement of the heart and includes transplanting the heart from another human or animal into the patient. The transplant procedure requires removing an existing organ (i.e. the natural heart) from the patient for substitution with another organ (i.e. another natural heart) from another human, or potentially, from an animal. Before replacing an existing organ with another, the substitute organ must be “matched” to the recipient, which can be, at best, difficult, time consuming and expensive to accomplish. Furthermore, even if the transplanted organ matches the recipient, a risk exists that recipient's body will still reject the transplanted organ and attack it as a foreign object. Moreover, the number of potential donor hearts is far less than the number of patients in need of a natural heart transplant. Although use of animal hearts would lessen the problem of having fewer donors than recipients, there is an enhanced concern with respect to the rejection of the animal heart.

In an effort to continue use of the existing natural heart of a patient, other attempts have been made to wrap skeletal muscle tissue around the natural heart to use as an auxiliary contraction mechanism so that the heart may pump. As currently used, skeletal muscle cannot alone typically provide sufficient and sustained pumping power for maintaining circulation of blood through the circulatory system of the body. This is especially true for those patients with severe heart failure.

Another system developed for use with an existing heart for sustaining the circulatory function and pumping action of the heart is an external bypass system such as a cardiopulmonary (heart-lung) machine. Typically, bypass systems of this type are complex and large, and as such are limited to short term use such as in an operating room during surgery or when maintaining the circulation of a patient while awaiting receipt of a transplant heart. The size and complexity effectively prohibit use of bypass systems as a long-term solution as they are rarely portable devices. Furthermore, long-term use of a heart-lung machine can damage the blood cells and blood borne products, resulting in post surgical complications such as bleeding, thromboembolism function, and increased risk of infection.

Still another solution for maintaining the existing natural heart as the pumping device involves enveloping a substantial portion of the natural heart, such as the entire left and right ventricles, with a pumping device for rhythmic compression. That is, the exterior wall surfaces of the heart are contacted and the heart walls are compressed to change the volume of the heart and thereby pump blood out of the chambers. Although somewhat effective as a short-term treatment, the pumping device has not been suitable for long-term use.

Typically, with such compression devices, a vacuum pressure is needed to overcome cardiac tissue/wall stiffness so that the heart chambers can return to their original volume and refill with blood. This “active filling” of the chambers with blood limits the ability of the pumping device to respond to the need for adjustments in the blood volume pumped through the natural heart, and can adversely affect the circulation of blood to the coronary arteries. Furthermore, natural heart valves between the chambers of the heart and leaching into and out of the heart are quite sensitive to wall and annular distortion. The movement patterns that reduce a chamber's volume and distort the heart was may not necessarily facilitate valve closure (which can lead to valve leakage).

Therefore, mechanical pumping of the heart, such as through mechanical compression of the ventricles, must address these issues and concerns in order to establish the efficacy of long term mechanical or mechanically assisted pumping. Specifically, the ventricles must rapidly and passively refill at low physiologic pressures, and the valve functions must be physiologically adequate. The mechanical device also must not impair the myocardial blood flow of the heart. Still further, the left and right ventricle pressure independence must be maintained within the heart.

Another major obstacle with long term use of such pumping devices is the deleterious effect of forceful contact of different parts of the living internal heart surface (endocardium), one against another, due to lack of precise control of wall actuation. In certain cases, this cooptation of endocardium tissue is probably necessary for a device that encompasses both ventricles to produce independent output pressures from the left and right ventricles. However, it can compromise the integrity of the living endothelium.

Mechanical ventricular wall actuation has shown promise, despite the issues noted above. As such, devices have been invented for mechanically assisting the pumping function of the heart, and specifically for externally actuating a heart wall, such as a ventricular wall, to assist in such pumping functions.

One particular type of mechanical ventricular actuation device that has been developed is a Left Ventricular Assist Device (LVAD), which is designed to support the failing heart. Such a device must augment systolic function. Diastolic function must also be augments or at the very least, not worsened, while allowing blood flow between the right and left ventricular portions of the heart. If the LVAD relies on a pump mechanism, the heart must still be able to beat 45 to 40 million times per year. The LVAD must therefore be durable and should function flawlessly or permit some degree of cardiac function in case of device failure. Such devices and/or systems must also permit a minimal risk for blood clot production and should be resistant to infection.

Other bodily functions rely on physical manipulation of muscles. For example, urinary and anorhectal sphincter valves control incontinence when operating properly. Sphincter valves are also founding the digestive tract where food passes from the esophagus into the stomach. Sphincter valves, however, tend to malfunction or lose range of operation, for example, after childbirth or as the human body ages. Surgery will sometimes correct incontinence in patients or reduce occurrences of Gastro esophageal reflux disease (GERD). Unfavorable conditions, however, often return or are sometimes not correctable using current treatments. Current artificial sphincter prototypes are composed of elastic and inflated with air. Erosion, probably from continuous high tonic pressure of inflated balloon in the urinary tract, can lead to infection and device failure. Therefore, there is a need for artificial means of restoring sphincter valve operation for digestive conditions. It is the inventors' belief that sphincter valve operation can be assisted or replaced using linear electromechanical systems.

Tendons are the thick fibrous cords that attach muscles to bone. They function to transmit the power generated by a muscle contraction to move a bone. Use of tendons can fail following trauma or because of arthritis. It is the inventors' belief that the movement of hands, fingers, arms, and legs that lose mobility can be assisted using linear electromechanical systems.

It is believed by the present inventors that a solution to the aforementioned problems associated with conventional ventricular assist devices and sphincter valves involves the use of linear electromechanical systems, such as linear actuators and linear motors. It is also believed that linear electromechanical systems can offer alternatives to other muscular dysfunctions encountered by patients due to age, disease or accidental causes.

A “linear motor” is essentially an electric motor that has had its stator “unrolled” so that instead of producing a torque (i.e., rotation) it produces a linear force along its length. Many designs have been put forward for linear motors, falling into two major categories, low-acceleration and high-acceleration linear motors. Low-acceleration linear motors are suitable for maglev trains and other ground-based transportation applications. High-acceleration linear motors are normally quite short and are designed to accelerate an object up to a very high speed and then release the object.

In most low-acceleration designs, the force is produced by a moving linear electromagnetic field acting on conductors in the field. Any conductor, be it a bop, a coil or simply a piece of plate metal, that is placed in this field will have eddy currents induced in the loop thus creating an opposing electromagnetic field. The two opposing fields will repel each other, thus forcing the conductor away from the stator and carrying it along in the direction of the moving magnetic field. Because of these properties, linear motors are often used in maglev propulsion, although they can also be used independently of magnetic levitation, as in the advanced light rapid transit technology such as that used in Vancouver's SkyTrain system, Toronto's Scarborough RT, New York City's JFK Airport AirTrain, and Kuala Lumpur's Putra LRT. Small-scaled versions of this technology are also used in robotics and manufacturing applications. The present inventors now believe that the current state of technology now makes it possible for “linear electromechanical” devices and systems such as linear motors, linear actuators and linear induction motors (LIMs) can be adapted for use in biomedical applications. Basic linear motor theory and functionality are well known. A reference book authored by Amitaca Basak entitled “Permanent-Magnet DC Linear Motors” (Clarendon Press—Oxford, 1996) in a solid survey of the subject matter that should already be fully understood by those skilled in the relevant art. Another textbook edited by E. R. Laithwaite entitled “Transport Without Wheels” (Eek Science—London, 1977) provides a useful compilation of papers contributed by authors familiar with transportation-related inear motion, which should also be familiar to the skilled.

### SUMMARY

The following summary of the invention is provided to facilitate an understanding of some of the innovative features unique to the embodiments and is not intended to be a full description. A full appreciation of the various aspects of the embodiments can be gained by taking the entire specification, claims, drawings, and abstract as a whole. The term “linear electromechanical” devices or systems should be read to include and define all possible linear electromagnetic manipulated devices that can be miniaturized to provide mechanical movement, including such devices as linear motors, linear actuators, linear induction motors, and other related linear devices known in the art, can now be adapted for use in biomedical applications.

It is a feature of the embodiments to provide linear electromechanical system for use to assist or replace human muscles, muscle/tendon operation, and sphincter valves.

It is another feature of the embodiments to provide a linear electromechanical device ventricular assist device.

It is another feature of the embodiments to provide a linear electromechanical device ventricular assist device in the form of at least one of: a cardial patch and a whole-heart wrap/jacket.

It is another feature of the embodiments to provide a linear electromechanical device based bio valve.

It is another feature of the embodiments to provide a linear electromechanical device bio valve that can be used as at least one of: an artificial anorectal sphincter, an artificial urinary sphincter, and an artificial gastroesophageal sphincter.

It is another feature of the embodiments to provide linear electromechanical device muscle and tendon operation within human extremities.

It is another feature of the embodiments to provide a linear electromechanical device muscle-tendon interface.

In accordance with more features of the embodiments, a system is described that includes a linear electromechanical device biological system interface, at least one sensor to monitor biological functions, a microprocessor for analyzing biological functions measured by the at least one sensor, a controller for causing operation of the linear electromechanical device to operate at least one of a ventricular assist device, bio valve and muscle-tendon interface, under direction of the microprocessor.

In accordance with more features of the embodiments, a system is described that includes integrated wire network provides sensory feedback, controlled contraction or relaxation of any single actuator or actuator groups, programmable contraction or expansion, and reflexic contraction or expansion from natural internal pacemakers.

In accordance with more features of the embodiments, a system is described that includes programmable contraction and expansion of artificial muscle regions and sub-regions, or artificial valves, programmable response to stimulus, and resistance to mechanical failure since multiple components operate in parallel.

It is yet a further aspect of the embodiments to provide for a ventricular assist device and system that is composed sheet of linear electromechanical device formed in/with material that can be wrapped around a failing heart to support ventricular activities thereof.

Additionally, linear electromechanical devices are linkable, contractile, durable and electrically insulated to performance characteristics by design. For example, a sheet can be configured from a flexible and/or a pliable material, and may be arranged as a sheath and/or in a mesh arrangement including linear electromechanical device.

The embodiments can be used for assistance of the following bodily functions/systems: Abdominal wall substitutes; Diaphragm substitutes; Artificial muscles such as skeletal muscle, Ocular muscle, Visceral muscle; Tendons as a muscle-bone interface; conduits; Sphincter Valves associated with reservoirs, the esophagus, prostrates, and the urinary bladder.

### BRIEF DESCRIPTION OF THE FIGURES

The accompanying figures, in which like reference numerals refer to identical or functionally-similar elements throughout the separate views and which are incorporated in and form a part of the specification, further illustrate at least one embodiment and, together with the detailed description of the invention, serve to explain the principles of embodiments,

FIG. 1 illustrates a heart with a mesh support system surrounding it for support in accordance with one embodiment;

FIG. 2 illustrates a pictorial perspective view of a human heart wherein ventricular activities can be supported and enhanced utilizing the embodiments;

FIG. 3 illustrates another pictorial perspective view of a human heart wherein ventricular activities are supported and enhanced utilizing another embodiment;

FIG. 4, labeled as “prior art,” illustrates a first type of linear actuator/motor known in the art for use in industrial applications;

FIG. 5, labeled as “prior art,” illustrates a second type of linear actuator/motor known in the art for use in industrial applications;

FIG. 6, labeled as “prior art,” illustrates the first type of linear actuator/motor shown in FIG. 4 moving from a passive mode at Time 1 to an actuated mode at Time 2;

FIG. 7, labeled as “prior art”, illustrates the second type of linear actuator/motor shown in FIG. 5 moving a shaft through electromagnets during electromagnet activation;

FIG. 8 illustrates more than one of the first type of linear actuator/motor from FIG. 4 connectable in a chain of linear electronmechanical devices integrated from housing to shaft throughout the chain in accordance with features of the invention;

FIG. 9 illustrates more than one of the of linear actuator/motor from FIG. 8 connected in a chain of linear electromechanical devices connected to a computer controlled power source in accordance with features of the invention;

FIG. 10 illustrates the plurality of electromagnets like that shown in FIG. 5, the electromagnets connected by hardware providing electrical power to the electromagnets, thereby linked together in a chain for moving the shaft with added power in accordance with features of the invention;

FIG. 11 illustrates a linear electromechanical device including an elongated housing surrounding electromagnetic material further surrounding a shaft movable with power provided to the electromagnetic housing by the computer and power source also illustrated, in accordance with features of the invention;

FIG. 12 illustrates a linear electromechanical system in accordance with features of the embodiments operating as a sphincter valve and including a microprocessor;

FIG. 13 illustrates another linear electromechanical system in accordance with features of the embodiments operating as a sphincter valve, and further shown in an “opened” position;

FIG. 14 illustrates a pictorial diagram of an artificial sphincter valve enabled in accordance with features of the embodiments in a “closed” position after having linear electromechanical assisted operation, and also shown is a sensor for monitoring bodily function in relation to operation of the sphincter valve;

FIG. 15 Illustrates a pictorial perspective view of a human hand and arrows indicating along the human hand where a linear electromechanical system in accordance with features that can be incorporated to provide muscle-tendon operation assistance;

FIG. 16 illustrates a pictorial perspective view of a human digestive system and arrows pointing to locations (e.g. esophagus, rectum, urinary tract) that artificial sphincter valves in accordance with embodiments;

FIG. 17 illustrates a pictorial perspective view of a human body and arrows pointing to locations (e.g., eyes, heart, esophagus, digestive tract, arms, legs, hands, feet) wherein linear electromechanical systems in accordance with features of the present invention can be employed, e.g., in the form of sphincter valves or muscle-tendon interfaces, in accordance with an alternate embodiment;

FIG. 18 is a flow diagram illustrating steps of how an electromechanical system in accordance with features of the present invention can operate autonomously within the human body, in accordance with an alternate embodiment; and

FIG. 19 illustrates a flow diagram showing steps wherein an electro-mechanical system operates within the human body in association with some human intervention, in accordance with an alternate embodiment.

### DETAILED DESCRIPTION

The particular values and configurations discussed in these non-limiting examples can be varied and are cited merely to illustrate at least one embodiment and are not intended to limit the scope thereof.

A natural human heart includes a lower portion comprising two chambers, namely a left ventricle and a right ventricle, which function primarily to supply the main pumping forces that propel blood through the circulatory system, including the pulmonary system (lungs) and the rest of the body, respectively. Hearts also includes an upper portion having two chambers, a left atrium and a right atrium, which primarily serve as entryways to the ventricles, and also assist in moving blood into the ventricles. The interventricular wall or septum of cardiac tissue separating the left and right ventricles is defined externally by an interventricular groove on the exterior wall of the natural heart. The atrioventricular wall of cardiac tissue separating the lower ventricular region from the upper atrial region is defined by atrioventricular groove on the exterior wall of the natural heart. The configuration and function of the heart is known to those skilled in this art.

Generally, the ventricles are in fluid communication with their respective atria through an atrioventricular valve in the interior volume defined by heart. More specifically, the left ventricle is in fluid communication with the left atrium through the mitral valve, while the right ventricle is in fluid communication with the right atrium through the tricuspid valve. Generally, the ventricles are in fluid communication with the circulatory system (i.e., the pulmonary and peripheral circulatory system) through semilunar valves. More specifically, the left ventricle is in fluid communication with the aorta of the peripheral circulatory system, through the aortic valve, while the right ventricle is in fluid communication with the pulmonary artery of the pulmonary, circulatory system through the pulmonic or pulmonary valve.

The heart basically acts like a pump. The left and right ventricles are separate, but share a common wall, or septum. The left ventricle has thicker walls and pumps blood into the systemic circulation of the body. The pumping action of the left ventricle is more forceful than that of the right ventricle, and the associated pressure achieved within the left ventricle is also greater than in the right ventricle. The right ventricle pumps blood into the pulmonary circulation, including the lungs. During operation, the left ventricle fills with blood in the portion of the cardiac cycle referred to as diastole. The left ventricle then ejects any blood in the part of the cardiac cycle referred to as systole. The volume of the left ventricle is largest during diastole and smallest during systole. The heart chambers, particularly the ventricles, change in volume during pumping. The natural heart, or rather the cardiac tissue of the heart, can fail for various reasons to a point where the heart can no longer provide sufficient circulation of blood from its operation so that bodily function and life can be sustained.

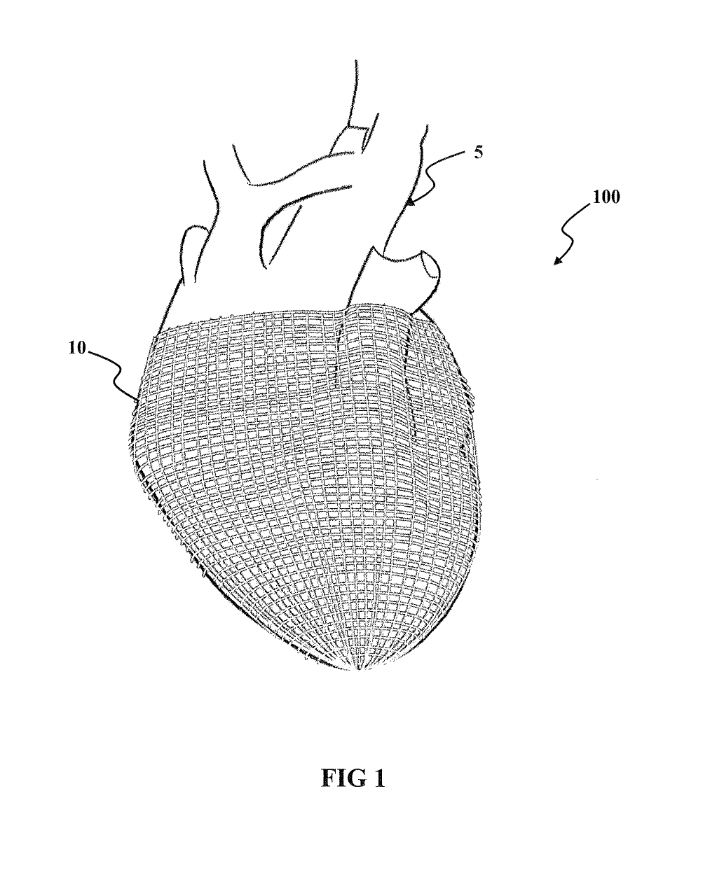

Referring to FIG. 1, a heart 5 is illustrated with a mesh support system 10 surrounding it like a jacket for support in accordance with embodiment of the present invention. The mesh-like sheet 10 can offer support to a failing heart so that it will not expand/swell, and can also include electromechanical operation within its grid-like structure (as will be further explained) in order to assist with pumping of the heart.

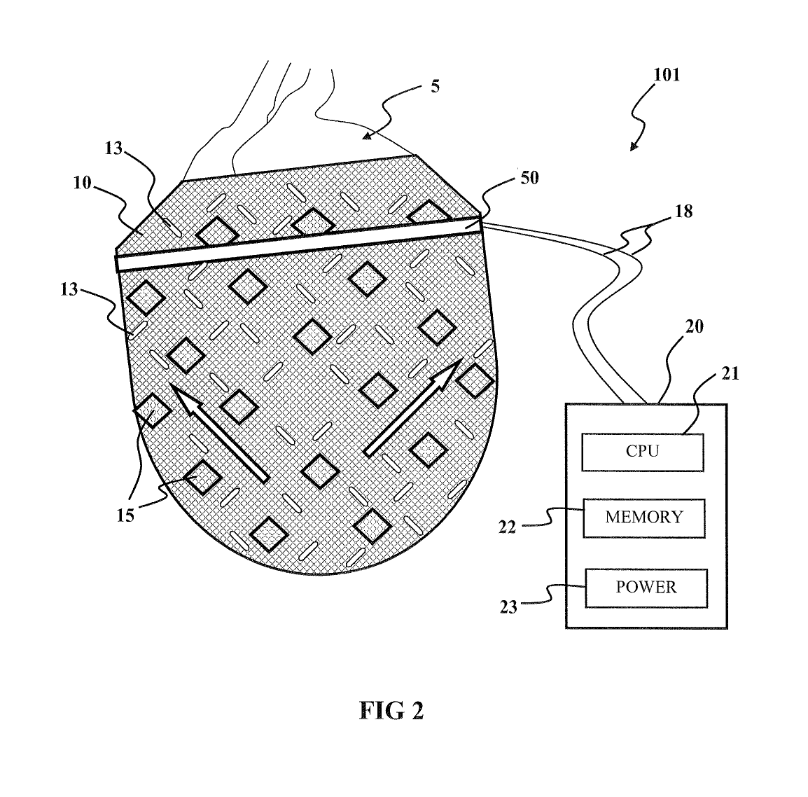

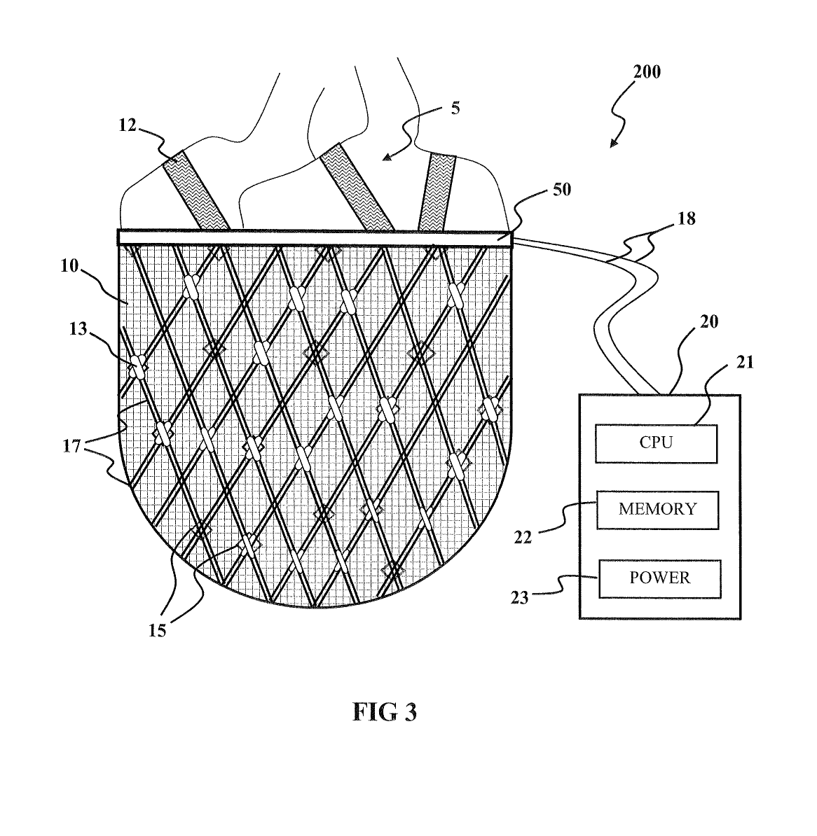

FIG. 2 illustrates a system wherein a biological function is controlled by a microprocessor and an electromechanical hardware implanted upon a biological system, in particular a human heart. The heart 5 is adapted with a mesh support system 10 including linear electromechanical devices 13 integrated with the mesh support system 10 providing compression action over chambers of the heart 5 in accordance with one embodiment of the present invention. Note that in FIGS. 2 and 3, identical or similar parts or elements are generally indicated by identical reference numerals. Thus, heart 5 depicted in FIG. 1 is also depicted in FIG. 2. Sheet 10 indicates wrapping of substantially all of the heart 5.

Illustrated in FIG. 2 are five general requirements including, as indicated above, that the electromechanically-based material of sheet 10 preferably includes linear electromechanical devices 13 either attached to the mesh, linked together over the mesh in a belt/band to other linear electromechanical devices 13 in the form of a belt or chain, or actuators can be linked together forming a jacket that is used around the heart in place of the mesh (e.g., operating also as the “mesh), as will be described in more detail below. As indicated by the large arrows on the mesh, each linear electromechanical device 13 integrated with the sheet 10 can cause compression of heart chambers after application of electrical current to the linear electromechanical device 13, which contributes to the generation of a force for contraction or expansion by sheet 10 in order to support natural ventricular activities of heart 5, which are believed to be similar to a wringing action by the muscles, and prevent failure thereof when sheet 10 is wrapped snuggly around heart 5.

Sheet 10 thus provides a contractile function. Relaxation can occur in the system by removing electrical current from linear electromechanical devices 13 during the hearts diastole status, or by allowing the heart muscles to expand into relaxed states between cycles while power is no longer applied. It should be appreciated that each linear electromechanical device among said plurality of elements composing sheet 10 is electrical insulated. As shown in FIG. 2, electrical contact to the linear electromechanical devices 13 can be facilitated between a controller 20 and a wiring located on the mesh 10 and contained and managed by band 50, which can operate as a conduit for electrical wires and feedback wiring 18. The wiring connects electrical DC current from a power supply 23 to electrical contacts (not shown) associated with each linear electromechanical device 13 composed of the mesh 10. It should be appreciated that a common ground can be provided using the mesh material, or separate contacts to each linear electromechanical device can be provided; however, it can be appreciated that less wiring is needed where a common ground is provided using mesh 10.

Also shown in FIG. 2 are sensors 15 integrated with and/or under the mesh 10. The sensor can monitor pressure created between the heart 6 and mesh 10. Results can be provided to the controller 20 where it can be analyzed by the CPU 21. The controller 20 can be provided as a self-contained module, similar to that provided with pacemakers. The controller 20 also provides power 23 to the mesh 10, sensors 15 and CPU 21. A memory 22 can be used to store results obtained from the sensor, and can also contained program instructions for the CPU 21 to use while operating the linear electromechanical devices 13 integrated with the mesh 10.

FIG. 3 illustrates a system 200 for assisting operation of a natural heart 5 in accordance with additional features of the embodiments. The system 200 still utilizes a controller 20, wiring 18, conduit 50 and mesh 10; however, the mesh 10 in FIGS. 1 and 2 no longer has linear electromechanical devices attached to or integrated therein to cause compression. Mesh 10 merely operates as a support material, like a stocking or a jacket, for the heart to prevent it from swelling. Linear electromechanical devices 13 and sensor 15 can be mounted on or over the mesh 10 on belts or bands 17. It is envisioned that moving shafts (not shown at this scale, but described below) of the mini-scaled linear electronmechanical devices 13 can be attached to the belts/bands 17 to cause compression of the heart over the mesh in accordance with the embodiments. The linear electromechanical devices 13 can be attached to the belts/bands 17 at junctions as shown in FIG. 3, which are illustrated at a criss-crossing of linear electromechanical actuators 13. The linear electromechanical actuators 13 in combination with the belts/bands 7 can cause compression and thereby pumping of the heart is directions indicated by the arrows illustrated in FIG. 2.

The electromechanical devices 13 can be integrated within the belts/bands 17 or firmly along the conduit 50 wherefrom the linear electromechanical devices 13 can pull on the belts/bands 17 in order to assist the heart with pumping. It can be noted that linear electromechanical devices 13 described with respect to FIG. 2 can also be accompanied by sensors 15 that can be deployed under the mesh 10. Also shown in FIG. 3 are support straps 12, which can be utilized to provide additional support to the mesh 10 and supported components (e.g., sensors 15 and devices (not shown)). Straps, like suspenders, can support the mesh 10 around most of the heart 5 and ensure pressure is applied against the heart by the electromechanical devices 13 via the bands 17 and mesh 10.

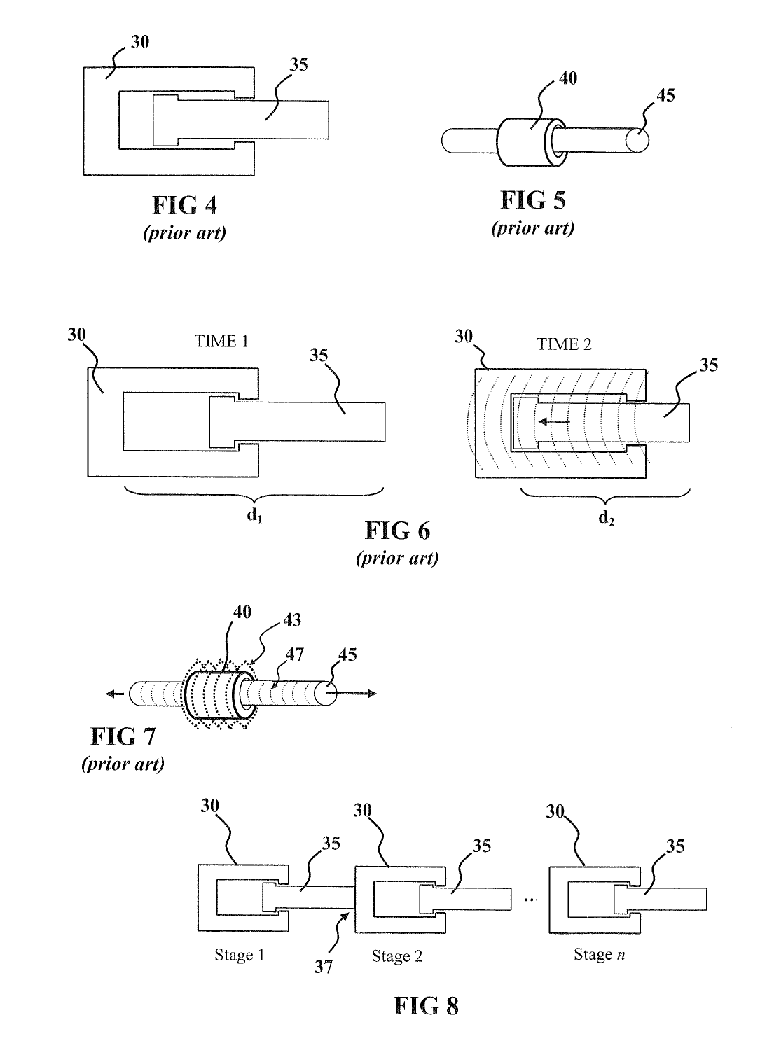

FIG. 4, which has been labeled as “prior art”, illustrates a basic linear DC motor or actuator. Such actuators are often used in small scaled applications related to robotics in support of semiconductor manufacturing, or can be used as the mechanism for moving a laser reader in a hard drive or DVD player. A linear actuator requires two basic components to operate, an electromagnet 30 and a shaft 35 that is reactive to electromagnetism. The electromagnet can also serve as the housing for the linear actuator.

FIG. 5, which has also been labeled as “prior art”, basic illustration of another linear motor/actuator. The linear actuator in this example can cause a shaft 45 to pass through the electromagnets 40 as power is applied to them. The electromagnets can be provided in the form of a coupler, or series of couplers, that can accept the shaft. A series of couplers of varying polarity action can be held together with hardware 40. The electromagnets can also be coated with a nonconductive material in a manner that presents the electromagnets as a unitary housing.

Referring to FIG. 6, labeled as “prior art”, the linear actuators illustrated in FIG. 4 is shown in action at Time 1 and Time 2. At time 1, no power has been applied to the electromagnetic housing 30 and the shaft 35 in fully extended from the housing in what can be termed a “relaxed state”. The distance d1 is shown for the relaxed states. At Time 2, power has been applied to the electromagnetic housing 30 as shown by magnetic waves. The electromagnetism caused by the application of power causes the shaft 35 to be drawn into the housing as indicated by the arrow located on the shaft. Once the shaft 35 is fully drawn into the housing 30, the distance d2 is created, which can be almost half the distance d1.

Referring to FIG. 7, labeled “prior art”, the linear electromechanical device of FIG. 5 is shown for operation in accordance with the embodiments. The electromagnetic housing 40 is provided in the form of a coupler adapted to receive a magnetically reactive shaft 45. When power is applied to the electromagnetic housing 40, electromagnetic current 43 causes the shaft made of magnetically reactive 47 materials to move through the couple in accordance with the magnetic fields induced thereon as shown by the arrows. Reversing polarity on the electromagnetic housing 40 can cause the shaft 45 to move in an opposite direction. Metal or iron shafts such as that shown in element 45 are well known. The metallic shaft can be produced with a solid or flexible material. It can be appreciated that the shaft can be coated to reduce friction as it interacts with the interior of the housing. The interior of the housing can also be coated in addition to or instead of the shaft. 45.

Referring to FIG. 8, linear actuators 30 are shown to be coupled together in stages 1-n. Each shaft 35 can be attached as indicated with area 37 to the housing 30 of another linear electromechanical device. Such a scheme can enable a larger distance of distance to be manipulated when the linear actuators are electrified. It can be appreciated that control of distance can be achieved by only activating one or a few of the linear actuators at a time. In an application as illustrated in FIGS. 2 and 3, shorting of a chain of linear actuators can help achieve compression over appropriate chambers of the heart 5.

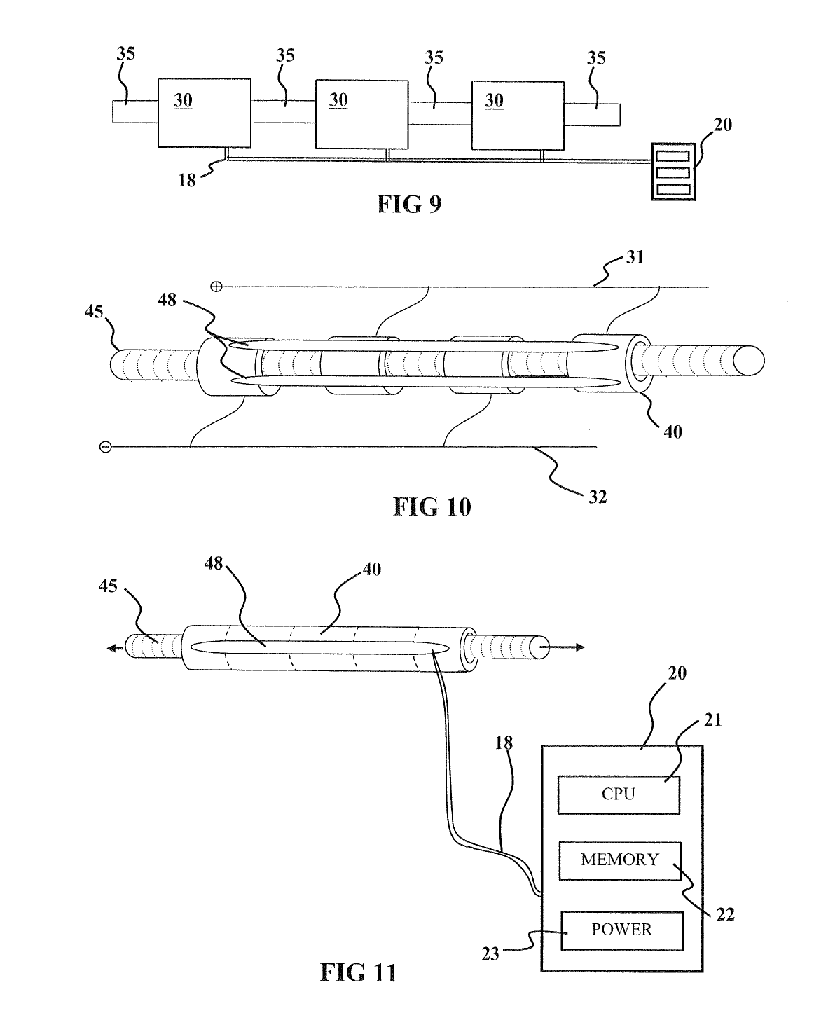

Referring to FIG. 9, what is illustrated are three linear actuators attached in a chain as described in FIG. 8. The linear actuators can be connected by wiring 18 and independently controlled by controller 20. The controller 20 can cause distance and power to each individual linear actuator to be monitored and controlled.

Referring to FIG. 10, four electromagnetic housings 40 are shown aligned and held together by hardware 48. A single shaft 45 is also shown in place through the center of the four electromagnetic housings 40. A controller 20 (as described above) can control the application of power to each electromagnetic housing 40, which in turn can affect the power and speed with which the shaft is moved through the electromagnetic housings 40. As can now be appreciated, more electromagnetic housings 40 working in parallel under the control of a controller 20 can enable a heart 5 to be efficiently assisted in its task of pumping blood through its chambers.

Referring to FIG. 11, a unitary housing 40 containing four partitioned electromagnetic coupling section is shown with a shaft 45 positioned therein for passage through the unitary housing based on electromagnetic force acting on the shaft from the unitary electromagnetic housing 40. Power and control wiring to each of the partitioned electromagnetic coupling sections can be provided from the controller 20 through a wiring conduit 48. It should be appreciated that the housing can be fabricated by layer of conductive materials with insulative barrier defining each zone. The insulting material can also be an adhesive between individual couplers, and a coating can be applied over the overall structure to create a unitary structure as shown.

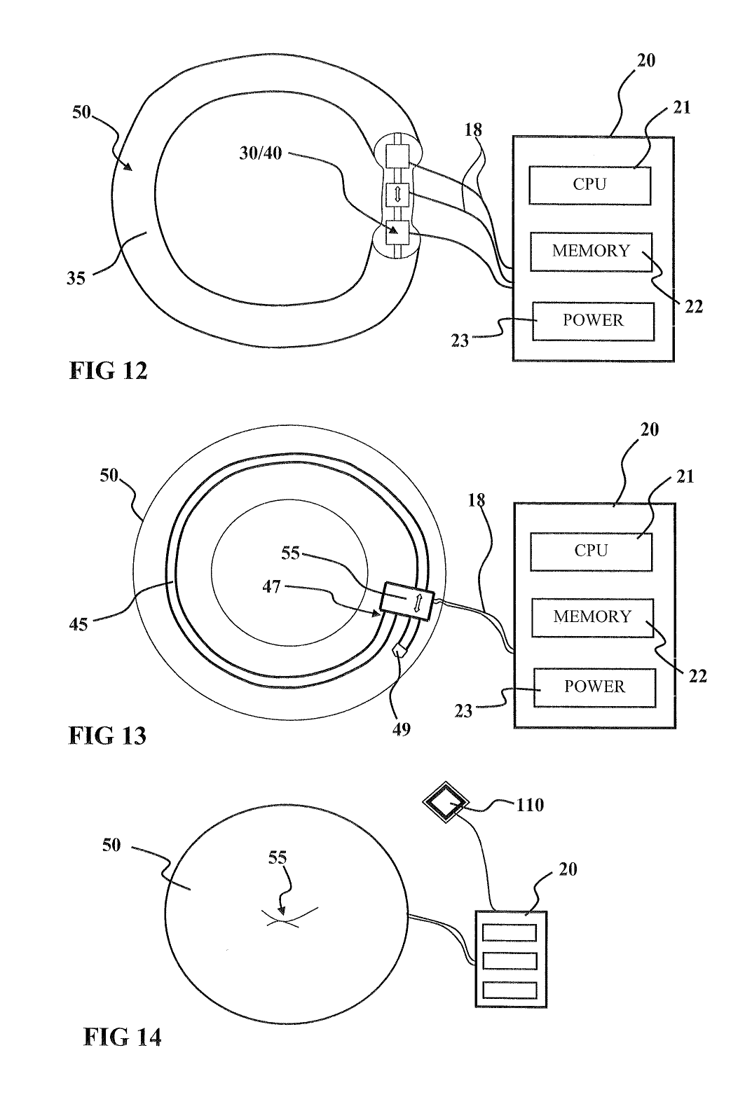

Referring to FIG. 12, shown is a donut-shaped device 50, which operates as a biological valve, such as a sphincter valve. The valve can be made of the tubular material 35. The tube-like structure can be provided in the form of an outer, insulative coating 35 that can protect the electromechanical contacts. The coating 35 should be flexible and compressible and should prevent the electromechanical hardware from interfering with the heart or other internal organs or tissue. The coating 35 also should prevent the system from shorting from exposure to bodily fluids. The coating can be made of a material (e.g. Gortex™) that is commonly used in surgical procedures with a purpose for lasting long durations in the body. The coating 35 cannot be easily rejected by the body and must be able to assimilate to the internal environment of the human body for relatively long periods of time. The sphincter valve is shown containing at least one linear electromechanical device 30/40 as part of an overall system 30. The linear electromechanical device(s) 30/40 is (are) wired 18 to a controller 20,

Referring to FIG. 13, another sphincter valve 50 is shown in accordance with carrying out the embodiments. A cross sectional view of the valve 50 is shown revealing the linear electromechanical device hardware inside. The linear electromechanical device includes an electromagnetic housing 55 with one end of a magnetically responsive shaft 45 (or belt) fixably attached to the housing as indicated at point 47. The magnetically responsive shaft 45 is set within and around the donut-shaped tubular material 50 until it meets up with and passes through an opening (not shown, but see FIGS. 7, 10 and 11) in the electromagnetic housing 55 where the shaft 45 then terminate at a tapered cap 22 near the shafts fixed location on the electromagnetic housing 40. When the electromagnetic housing 40 is provided power, electromagnetic field acting on the shaft 45 where it is located within the opening cause the shaft to move through the opening, which then causes the diameter of the circle created by the shaft to become smaller. When the shaft and housing are placed in a donut-shaped tubular material 50, the donut-shaped tubular material 50 will also shrink in diameter with movement of the shaft 45. The controller 20 will apply controlled power to the electromagnetic housing 40 though wiring 18.

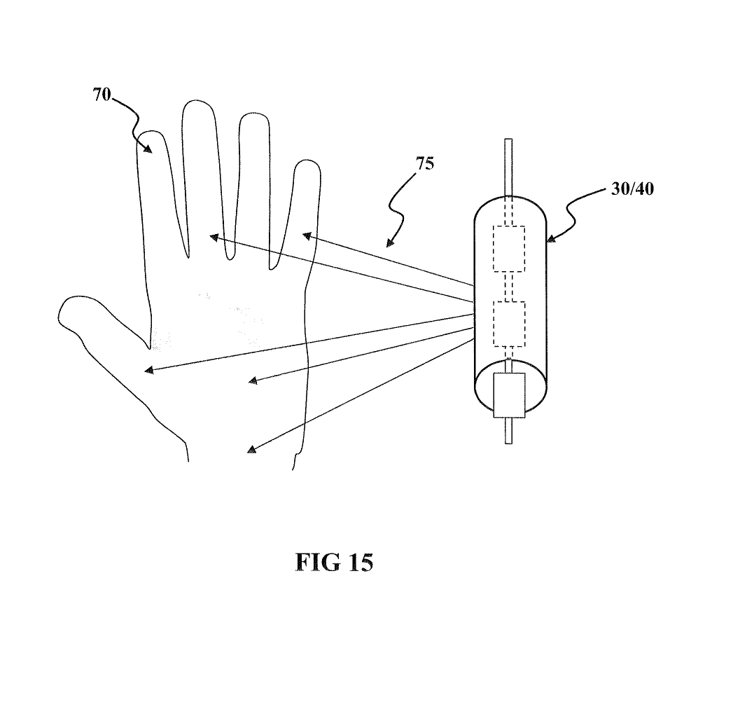

Referring to FIG. 14, the donut-shaped tubular material 50 is shown in a dosed position as indicated at location 55, which can be achieved by movement of the shaft 45 through the electromagnetic housing 40 under control of the controller 20. Also shown associated with the bio valve 105 is a sensor 15. The sensor 15 and controller 20 can be programmed to cause the bio valve 105 to open or dosed in accordance with a specific application or manipulation. For example, closure (tightening) of the valve 55 can be caused by the electromechanical system contained inside the tubular shape of the valve can be caused by an electronic signal. The signal can be based on sensor 15. For example, if the valve 50 is being used as the sphincter valve between the esophagus and the stomach, then GERD can be prevented when a patient is not eating and the valve remains dosed. But if the sensor sensing food passing through the esophagus, the valve 50 can be caused to open (relax). It can be appreciated that a design is possible where the valve can remain normally dosed unless a sensor causes the application of power to loosen the valve by causing the shaft to travel in an opening direction.

When a sensor 15 located above the sphincter valve 50 can be activated because it senses food traveling into the esophagus, then the valve is cause to relax or open. The sensor can be a pressure transducer, electrical contact sensor, or electro-impulse detector. A pressure transducer can sense the weight of food or water within the esophagus above the valve. It can now be appreciated that a similar sensor-valve configuration can be employed in other parts of the human body. For example, the sphincter valve 50 can be implanted in a patient's rectum or after the bladder. The valve can help patient control incontinence. Such an application would be helpful for cancer patients that have lost functionality due to rectum or prostrate cancer, or adults that can no longer control urinary function because of age or numerous childbirths. It is also possible that the sensor can be in communication with the nervous system for receiving signaling associated with performing a specified function (e.g., opening the sphincter valve).

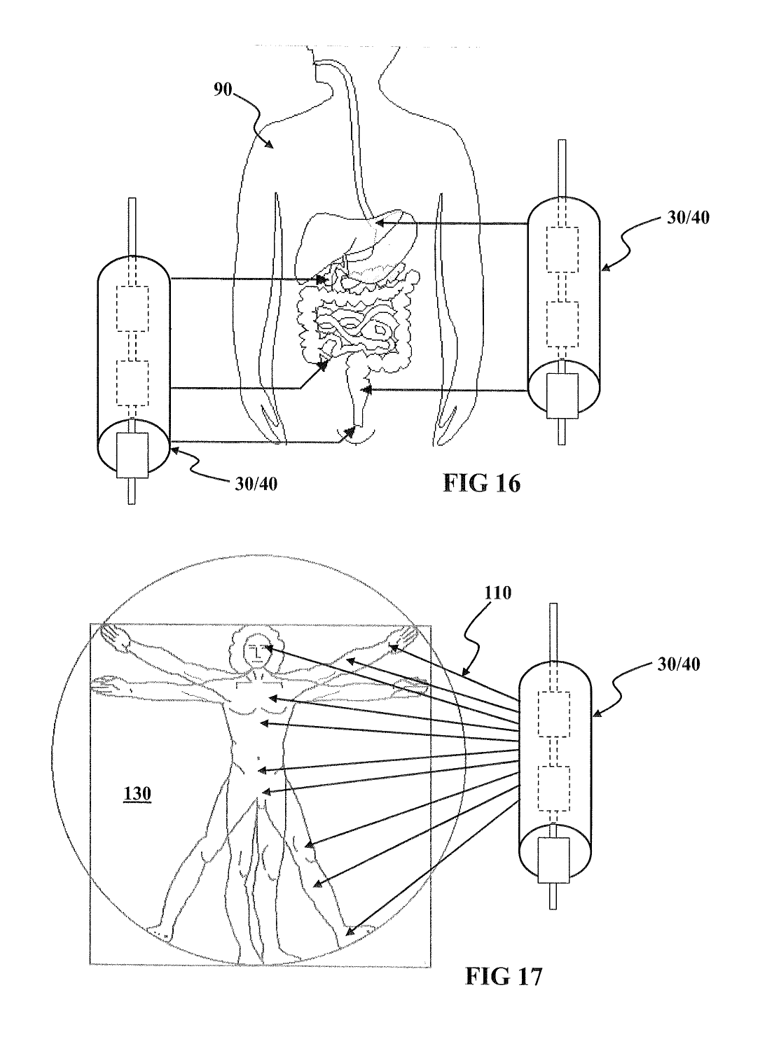

FIG. 15 illustrates a hand 70 with arrows 75 pointing from an electromechanical system 30/40 to areas on the hand where mechanical function may be of help. Tendons in hands, feet, arms legs, etc., may no longer function well because of arthritis or because of nerve loss. It can now be appreciated following this description that linear electromechanical systems can be devices to assist in the movement of tendons by muscles located within a body's extremities. Referring to FIG. 16, a patient 90 is shown with arrow pointing to areas within the digestive tracts wherein electromechanical systems 30/40 may assist with control functions. Referring to FIG. 17, a human body 130 is shown with arrows 110 pointing to location on the body where electromechanical systems 30/40 may assist with bodily movement.

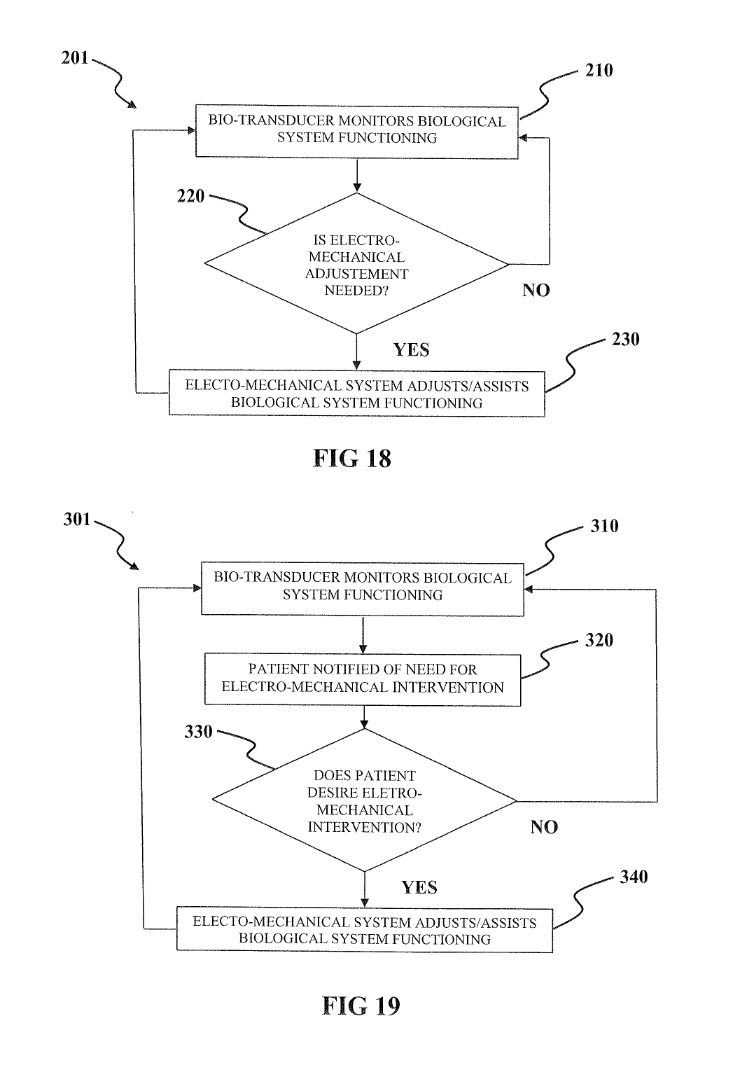

Referring to FIG. 18, a flow diagram 201 is shown including steps of linear electromechanical system function in the human body. A controller/monitor, similar to the controller 20 and sensors 15/110 previously described can carry out the following steps. As shown in block 210, a bio-transducer monitors biological system functioning. As shown in decision block 220, the system inquires whether electroemchanical adjustment is needed. If not, the process returns/maintains monitoring status of block 210. If adjustment is necessary, the as shown in block 230, a linear electromechanical system adjusts/assists a biological system with functioning. It can now be appreciated that the monitoring can cause operation where, for example, food is sensed in the esophagus, or when the heart requires faster/slower operation based on load requirements of the patients (e.g., exercise, or rest).

Referring to FIG. 19, a flow diagram 301 is shown where patient intervention can be allowed to a system. As shown in block 310, a bio transducer monitors a biological system's functioning. As shown in block 320, a patient can be notified of a need for electro-mechanical intervention. Notification can occur, for example, where the patient is exerting himself and requires faster pumping of the heart, or when a sensor indicates (e.g., vibrates, alarms, or other sensation) that a valve must be operated. As shown in decision block 330, the system is waiting for input by a patient as to whether electro-mechanical intervention is needed. If not, then monitoring continued in block 310. If intervention is requested, then the electromechanical system can cause adjustments or assistance of a biological system for occur as thought herein.

A controller 60 is generally in communication with said plurality of linear electromechanical elements 30/40, while a microprocessor 90 is generally in communication with controller 60. Microprocessor 90 and controller 60 can be implemented in the context of a pacemaker 90, which is generally in communication with electrical devices. Microprocessor 90 can be implemented as a central processing unit (CPU) on a single integrated circuit (IC) computer chip. Microprocessor 90 generally functions as the central processing unit of apparatus 70, can interpret and execute instructions, and generally possesses the ability to fetch, decode, and execute instructions and to transfer information to and from other resources over a data-transfer path or bus.

Note that each linear electromechanical element among said plurality of linear electromechanical elements can contract toward one another in systole and away from one another by a reversal of poles in diastole. Additionally, each electromechanical element among said plurality of electromechanical elements will preferably sequentially contract the heart horizontally and thereafter, vertically. As indicated previously, each electromechanical element is electrical insulated. Sheet 10 can be configured from a flexible or pliable material. Tube 35 can be configured from a flexible or pliable material.

Unique features of the linear electromechanical-biological devices and systems described herein includes: integrated wire network, sensory feedback, controlled contraction or relaxation of any single actuator or actuator groups, programmable contraction or expansion, reflexic contraction or expansion from natural internal pacemakers, programmable contraction and expansion of any regions and sub-regions, programmable response to stimulus, resistance to mechanical failure since multiple components operate in parallel or over a grid configuration.

As a cardiac patch, the present invention offers a simpler design than a whole-heart wrap design and can be used to target a specific location of failure along an organ. The cardiac patch can be surgically affixed to cardiac regions and surfaces along a heart. For example, a patch can be placed over area of myocardial scar, aneurysm, or defect. The patch is sutured in place over the afflicted area. The electromechanical system within the patch can be programmed to contract and expand with heart cycles that are being sensed using sensors located near or within the patch and monitored by a microprocessor. Using this configuration, sub regional contraction and expansion is optimized with external programming and radiologic real-time visualization. Other advantages of the patch system are that it provides self-contractile material to reinforce weakened or absent myocardium. The externally applied patch need not contact blood. Coagulation problems are avoided. Surgical excision of defective tissue is avoided.

Because artificial Anorectal Sphincters are desperately needed by fecal incontinence patients (stomates patients with a surgically removed rectum or anus and a diverting colostomy). An electromechanical system can be surgically implanted to surround native anorectum or surgically translocated conduit (colon pulled into place formerly occupied by the anorectum). Baseline conformation is relaxation of upstream canal and relative contraction of downstream canal. Manual switch activation or direct signal transduction from the sacral and inferior hemorrhoidal nerves allows defecation by stimulating upstream canal contraction and downstream canal relaxation. Reflex continence is maintained when the switch is not activated or by voluntary impulses, in these conditions, propagating impulses sensed from upstream bowel produce a reflex increased capacitance of the upstream sleeve and temporary hypercontraction of the downstream sleeve. A relatively thin artificial sphincter assist produces a programmable limit of pressure on tissue.

Now, an artificial Urinary Sphincter can be provided in accordance with feature of the present invention to prevent urinary Incontinence caused by female stress or side affects of male surgery for prostrate issues. An Artificial Gastroesophageal Sphincter provided utilizing features of the present invention can prevent gastroesophageal reflux. A cylindrical tube including electroemchanical functioning can be surgically implanted to fit around the gastroesophageal junction in a patient. Relatively contracted in baseline conformation to prevent gastroesophageal reflux. The Artificial Gastroesophageal Sphincter of the present invention is induced to relax by sensed distension of upstream esophagus. Anti-reflux prosthetic devices of the past (e.g., Angelchick prosthesis) can now be abandoned because of prior problems with prosthesis migration or erosion.

The embodiments and examples set forth herein are presented to best explain the present invention and its practical application and to thereby enable those skilled in the art to make and utilize the invention. Those skilled in the art, however, will recognize that the foregoing description and examples have been presented for the purpose of illustration and example only. Other variations and modifications of the present invention will be apparent to those of skill in the art and it is the intent of the appended claims that such variations and modifications be covered.

The description as set forth is not intended to be exhaustive or to limit the scope of the invention. Many modifications and variations are possible in light of the above teaching without departing from the scope of the following claims. It is contemplated that the use of the present invention can involve components having different characteristics. It is intended that the scope of the present invention be defined by the claims appended hereto, giving full cognizance to equivalents in all respects.

1. A linear electromechanical device, comprising:
electromagnetically actuated hardware including at least one linear electromechanical device that further comprises a flexible shaft and is assembled into a circle and is surrounded by the protective coating, and the flexible shaft formed in a circle is used as a bio valve adapted for use in a biological system to replace or supplement operation of a biological valve;
a protective coating surrounding the electromagnetically actuated hardware and acting as a barrier between the electromechanically actuated hardware and biological systems;
at least one sensor to monitor biological system functions;
a microprocessor analyzing biological system functions measured by the at least one sensor; and
a controller causing operation of the electromechanically actuated system to operate under direction of the microprocessor as a biological valve.

2. The system of claim 1 wherein the controller is programmed to cause the electromechanically actuated hardware to cause contraction or expansion of a biological system.

3. The system of claim 1 wherein the electromagnetically actuated hardware comprises more than one linear electromechanical device connected in a chain.

4. The system of claim 3, wherein the controller is programmed to cause the electromechanically actuated hardware to cause contraction or expansion of a biological system.

5. The system of claim 1, wherein said flexible shaft is assembled into a circle is used as a sphincter valve replacement within a human body.

6. The system of claim 2, wherein said flexible shaft is assembled into a circle is used as a sphincter valve replacement within a human body.

7. The system of claim 3, wherein said flexible shaft is assembled into a circle is used as a sphincter valve replacement within a human body.

8. The system of claim 4, wherein said flexible shaft is assembled into a circle is used as a sphincter valve replacement within a human body,

9. An apparatus for assisting biological system functions, the apparatus comprising:
a controller in communication with a linear electromechanical device including more than one linear motor assembled as at least one chain link, wherein the chain link shortens as power is applied to the more than one linear motor and the chain link expands when power is no longer applied to the more than one linear motor, wherein the chain link formed in a circle is used as a biological valve adapted for use in a biological system to replace or supplement operation of the biological valve; and
a protective coating surrounding the linear eletromechanical device and acting as a barrier between the electromagnetically actuated hardware and biological systems.

10. The apparatus of claim 9, further comprising:
at least one sensor to monitor biological system functions; and
a microprocessor analyzing biological system functions measured by the at least one sensor.

11. The system of claim 10, wherein the controller is programmed to cause the electromechanically actuated hardware to cause contraction or expansion of a biological system.

12. The system of claim 9, wherein said flexible shaft is assembled into a circle is used as a sphincter valve replacement within a human body.

13. The system of claim 10, wherein said flexible shaft is assembled into a circle is used as a sphincter valve replacement within a human body.

14. The system of claim 11, wherein said flexible shaft is assembled into a circle is used as a sphincter valve replacement within a human body.

15. A linear electromechanical system, comprising:
at least one linear electromechanical device including more than one linear motor assembled as at least one chain link, wherein the chain link shortens as power is applied to the more than one linear motor and the chain link expands when power is no longer applied to the more than one linear motor, wherein the chain link formed in a circle is used as a biological valve adapted for use in a biological system to replace or supplement operation of the biological valve;
a protective coating surrounding the at least one linear electromechanical device and acting as a barrier between the at least one linear electromechanical device and biological systems; and
a microprocessor causing the at least one linear electromechanical device to operate as a biological valve.

16. The system of claim 15, wherein the at least one linear electromechanical device includes more than one linear actuator assembled as at least one chain link wherein positive and ground connections are connected to the more than one linear actuator forming the at least one chain link, wherein the chain link shortens as power is applied to the more than one linear actuator and the chain link expands when power is no longer applied to the more than one linear actuator,

17. The system of claim 15, including a microprocessor, wherein the microprocessor is programmed to cause the electromechanically actuated hardware to cause contraction or expansion of at least one sphincter valve.

18. The system of claim 16, wherein said chain link assembled into a circle is used as a sphincter valve replacement within a human body.

19. The system of claim 16, wherein the circumference of the circle shortens as power applied to the chain link to and the circumference of the circle lengthens when power is no longer applied to the chain link.

20. The system of claim 19, wherein said chain link assembled into a circle is used as a sphincter valve replacement within a human body.
